(19) United States
(12) Patent Application Publication
(10) Pub. No.: US 2005/0107877 A1
(43) Pub. Date: May 19, 2005

(54) System and methods for restoring the structural integrity of bone

(75) Inventor: Jason Blain, San Diego, CA (US)

Correspondence Address: ATTN: LEGAL DEPARTMENT;NU VASIVE, INC., 4545 TOWNE CENTRE COURT, SAN DIEGO, CA, 92121, US

(73) Assignee: Nu Vasive, Inc., San Diego, CA (US)

(21) Appl. No.: 10/979,932

(22) Filed: Nov. 1, 2004

Related U.S. Application Data
(60) Provisional application No. 60/516,946, filed on Oct. 30, 2003.

(51) Int. Cl. A61F 2/28; A61F 2/44

(52) U.S. Cl. 623/16.11; 623/17.11

(57) ABSTRACT

A laminoplasty device including a superior attachment panel, inferior base panel, and a coupling screw for use in repair of lamina after spinal surgery. The interior of the fully assembled laminoplasty device contains a fusion corridor in which bone fusion material can be placed to facilitate regeneration of the lamina.

### CROSS REFERENCES TO RELATED APPLICATIONS

The present application is a nonprovisional patent application claiming benefit under 35 U.S.C. § 119(e) from U.S. Provisional Application Ser. No. 60/516,946, filed on Oct. 30, 2003, the entire contents of which are hereby expressly incorporated by reference into this disclosure as if set forth fully herein.

### BACKGROUND OF THE INVENTION

I. Field of the Invention

The present invention relates generally to restoring the structural integrity of bone and more particularly, to repair and fusion of the lamina after spinal surgery.

II. Discussion of the Prior Art

Spinal stenosis is a condition in which the spinal canal has been narrowed or structured, and although it is a problem that occurs mostly in the aging population, it can also affect younger persons who have experienced certain types of trauma. The causes of spinal stenosis are many, but a partial list includes congenital stenosis, bone spurs, intervertebral disc herniation, ossification of the posterior longitudinal ligament or ligamentous flavum, and spondylosis. Often times patients with spinal stenosis, particularly the congenital type, will experience no symptoms until later in life. However, stenosis can cause myelopathy (a disease of the tissue surrounding the spinal cord) and myeloradiculopathy (disease of the spinal cord and/or nerve roots). Patients with either of these disorders may experience a wide range of symptoms, including pain in the upper body region (especially in cases of myeloradiculopathy), weakness in the hands, spastic or clumsy gait due to weakness in legs, and possibly urinary disorders, particularly a constant feeling of urgency.

As a result of these spinal disorders, many patients experience a serious reduction in the ability to carry on everyday activities, and thus the problem must be treated. For people in which symptoms of stenosis are not as severe, or there is little to no pain involved, often times surgery is not required. These patients would be subject to conservative, non-surgical methods of treatment. For cervical stenosis, this usually involves wearing a rigid neck brace at night, and a soft brace during the day, so as not to impede normal activities more than is necessary. Other types of conservative treatment for cervical myelopathy include analgesia, physical therapy, and anti-inflammatory/steroid treatment. Conservative treatment for patients suffering from lumbar stenosis includes physical therapy and medication. If conservative treatment is unsuccessful, then surgery must be considered.

The traditional surgical procedure for such a condition is laminectomy, or open decompression. In a laminectomy, the sipnous process and a portion of the lamina over the nerve root is removed in order to alleviate the pain. While this operation can be successful in solving the immediate problem (pain in the back, arms, or legs), the procedure itself creates its own set of problems. Removal of the lamina and sipnous process can weaken the stability of the spinal column, especially in elderly patients. Patients undergoing cervical laminectomy may experience postoperative kyphosis, or swan neck deformity, due to the removal of bone structure. Finally, many laminectomy patients develop a post-laminectomy membrane, or a formation of scar tissue involving the nerve root and surrounding structures. This is especially unfortunate because the scar tissue in effect puts pressure on the nerve that the surgical procedure was designed to alleviate.

Another procedure used to alleviate nerve pinching is anterior cervical decompression and spinal fusion (ACDF). This procedure is used when a herniated disc is the cause of back pain. In this procedure, the offending disc is removed, thereby alleviating the pain. However, since the disc performs some structural function, spinal fusion is also employed to maintain the spatial integrity of the spinal column. As such, the spine becomes more rigid depending on how many discs need to be removed (and consequently how many vertebra need to be fused together). Patients undergoing ACDF may experience temporary hoarseness and difficulty swallowing due to irritation of the nerve leading to the vocal cords (recurrent laryngeal nerve) and esophagus caused by retraction during surgery.

As a result of problems associated with laminectomy and ACDF, the laminoplasty was developed and is being used with increasing regularity. The primary advantages of laminoplasty are that it avoids complications associated with destabilization and immobilization of the spine, and eliminates the danger of post-laminectomy kyphosis and the post-laminectomy membrane. In a laminoplasty, the lamina on one side of the sipnous process is cut, but not removed. On the other side of the sipnous process, a groove is formed such that the lamina can swing posteriorly in a hinge-like manner. As a result of this, the procedure is often called “open-door laminoplasty.” The lamina can swing outward, creating more space in the spinal canal, thereby alleviating the pain caused by spinal stenosis.

Because the lamina is not removed and the structural integrity of the spinal canal is intact, there is no need to fuse the vertebrae together. However, because one side of the lamina has been severed, there may be a problem with stability of the spine at that juncture. Thus, many techniques have developed in order to try to repair the lamina after laminoplasty. One such technique is to use a bone graft from another place, usually the hip, to act as a wedge between the open ends of the lamina. However, this method is unstable and can lead to slippage of the bone graft. Commonly, the ends of the severed lamina are held together with a wire device until the bone can regenerate itself, but this is also relatively unstable and can take time.

The present invention is directed at overcoming, or at least improving upon, the disadvantages of the prior art.

### SUMMARY OF THE INVENTION

The present invention accomplishes this goal by providing a stabilizing device that encourages reformation of the lamina in its new position.

According to one broad aspect of the present invention, the laminoplasty device comprises a superior attachment panel and an inferior base panel that are attached to each side of the laminal opening and are held together by a screw or bolt. When secured into place, the device forms an interior tunnel in which fusion material may be placed in order to regenerate the lamina.

The superior attachment panel may include any number of components capable of creating a bridge over the post-laminectomy gap. By way of example only, the superior attachment panel may be composed of ceramic, steel, plastic, bone, or any other biocompatible material. The superior attachment panel may be shaped as a square, rectangle, or any other shape necessary to effectively bridge the gap. Preferentially, the superior attachment panel should contain a bore in the central region of the latitudinal surface, for the purpose of inserting a coupling screw. In addition to the coupling screw, the superior attachment panel may also contain any number of means of attachment to the inferior base panel, including but not limited to pegs of varying number, placement, length, and diameter. In a preferred embodiment, the bore may be threaded, but it is not necessary. The corners of the inferior face of the superior attachment panel may contain any number of means of attachment to the lamina to prevent slippage of the device. By way of example only, the attachment mechanism may consist of spikes or pegs, but a corrugated surface is also contemplated.

The inferior base panel may include any number of components capable of creating a bridge over the post-laminectomy gap. By way of example only, the inferior base panel may be composed of ceramic, steel, plastic, bone, or any other biocompatible material. The inferior base panel may be shaped as a square, rectangle, or any other shape necessary to effectively bridge the gap, but should be in the same shape as the superior attachment panel. Preferentially, the inferior base panel should contain a threaded bore in the central region of the latitudinal surface, for the purpose of receiving a coupling screw. In addition to the coupling screw, the inferior base panel may also contain any number of means of attachment to the superior attachment panel, including but not limited to pegs of varying number, placement, length, and diameter. The corners of the superior face of the inferior base panel may contain any number of means of attachment to the lamina to prevent slippage of the device. By way of example only, the attachment mechanism may consist of spikes or pegs, but a corrugated surface is also contemplated.

The fusion material may include any number of materials capable of facilitating fusion or regeneration of the lamina bone. By way of example only, the fusion material may include bone grafted from another part of the body, including but not limited to the hip bone.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIGS. 1-2 are exploded perspective and perspective views, respectively, of a laminoplasty device according to the present invention;

FIG. 3 represents the laminoplasty device of the present invention as finally inserted into the human vertebrae;

FIGS. 4-6 illustrate alternative embodiments of the laminoplasty device of the present invention.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The system and methods for restoring the structural integrity of bone disclosed herein boasts a variety of inventive features and components that warrant patent protection, both individually and in combination.

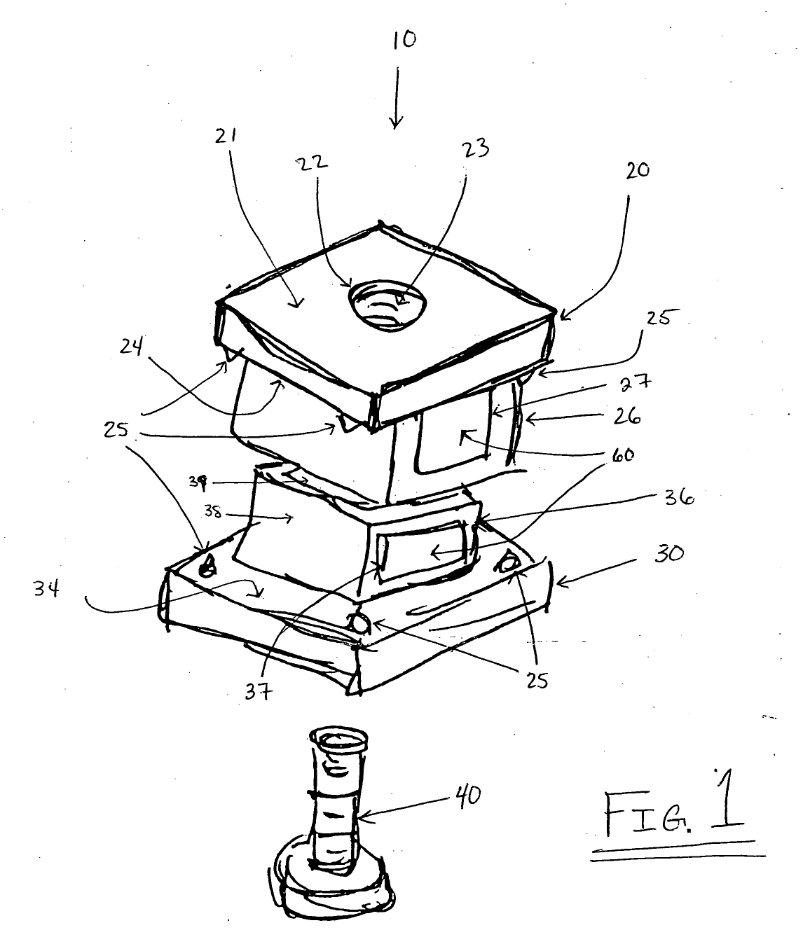

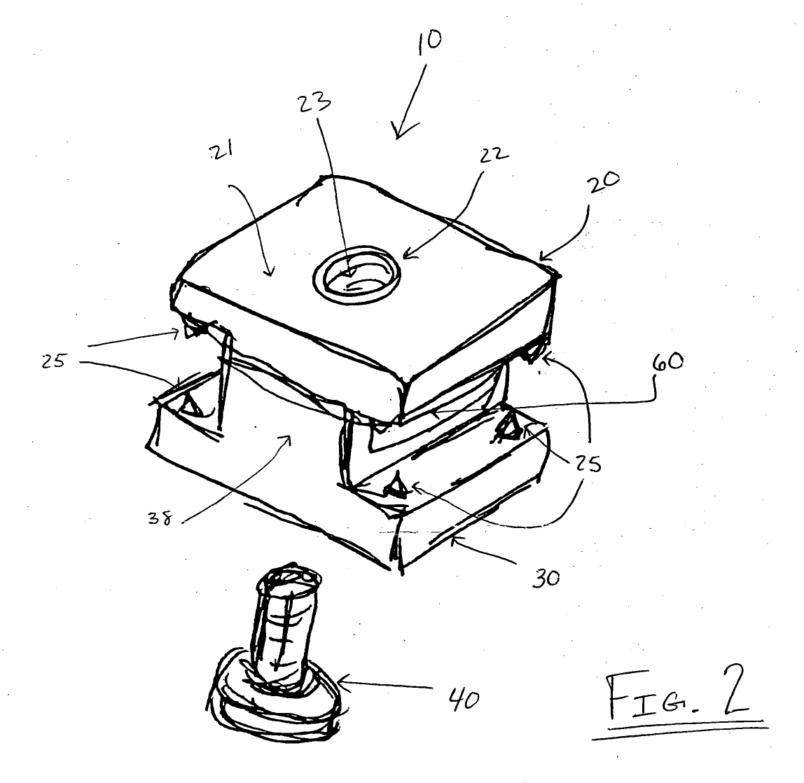

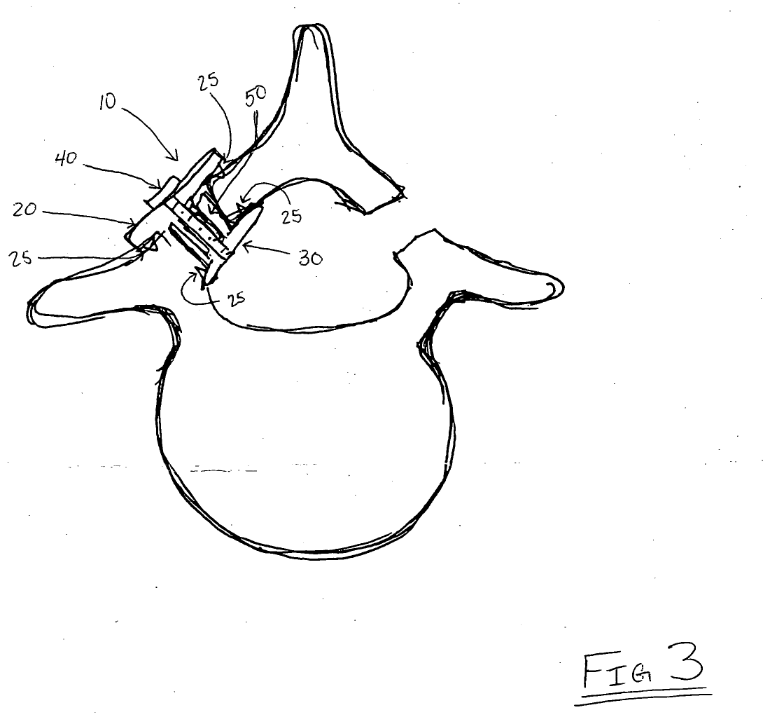

FIGS. 1-2 illustrate a preferred embodiment of the laminoplasty device 10 of the present invention. The laminoplasty device 10 includes a superior attachment panel 20, an inferior base panel 30, and a coupling screw 40. The superior attachment panel 20 is situated on one side of the lamina (See FIG. 3), and includes a bore 22 that, in a preferred embodiment, includes threads 23. Bore 22 is located in a relatively central location of superior latitudinal surface 21 of superior attachment panel 20. Through bore 22 passes coupling screw 40, which when attached with inferior base panel 30 will securely hold together the fully assembled laminoplasty device 10. Inferior latitudinal surface 24 of superior attachment panel 20 contains spikes 25 located, in a preferred embodiment, at each of four corners of superior attachment panel 20. It is contemplated that in alternative embodiments of laminoplasty device 10, in which the shape of superior attachment panel 20 is other than rectangular or square, spikes 25 may be located in positions advantageously dictated by the shape of the panel.

Attached to and extending perpendicularly from inferior latitudinal surface 24 of superior attachment panel 20 is the exterior fusion corridor housing 26. Exterior fusion corridor housing 26 is a four-sided structure comprised of two opposing lateral corridor windows 27 and two opposing solid planar walls 28. Superior attachment panel 20 forms the ceiling of the exterior fusion corridor housing 26, while the floor is hollow to accommodate coupling with interior fusion corridor housing 36 and to allow coupling screw 40 to pass through. Shown in a square or cubic orientation, it is contemplated that exterior fusion corridor housing 26 could maintain a rectangular or other shape determined in part by the shape of superior attachment panel 20.

Inferior base panel 30 is situated on the other side of the lamina (See FIG. 3), and includes a threaded bore 32 (not shown). Threaded bore 32 is located along the same longitudinal plane as bore 22 of superior attachment panel 20, in order to effectively accommodate coupling screw 40. Through bore 32 passes coupling screw 40, which when attached with superior attachment panel 20 will securely hold together the fully assembled laminoplasty device 10. Superior latitudinal surface 34 of inferior base panel 30 contains spikes 25 located, in a preferred embodiment, at each of four corners of the inferior base panel 30. It is contemplated that in alternative embodiments of laminoplasty device 10, in which the shape of the inferior base panel 30 is other than rectangular or square, spikes 25 may be located in positions advantageously dictated by the shape of the panel.

Attached to and extending perpendicularly from superior latitudinal surface 34 of inferior base panel 30 is the interior fusion corridor housing 36. The interior fusion corridor housing 36 is a four-sided structure comprised of two opposing lateral corridor windows 37 and two opposing solid planar walls 38. Inferior base panel 30 forms the floor of interior fusion corridor housing 36, while the ceiling contains a vertical corridor window 39 to allow coupling screw 40 to pass through. Shown in a square or cubic orientation, it is contemplated that interior fusion corridor housing 36 could maintain a rectangular or other shape determined in part by the shape of inferior base panel 30 and exterior fusion corridor housing 26. When laminoplasty device 10 of the present invention is fully assembled, interior fusion corridor housing 36 will fit securely inside exterior fusion corridor housing 26, forming the completed fusion corridor 60, and allowing fusion material 50 (not shown) to fuse to the lamina and regenerate a solid bone structure (See FIG. 2).

FIG. 3 shows the fully assembled laminoplasty device 10 as it would appear installed in a human vertebra following laminoplasty surgery. Spikes 25 engage remaining lamina on either side of the gap, holding laminoplasty device 10 in place. Coupling screw 40 can be seen to extend completely through laminoplasty device 10, originating at bore 22 (not shown) of superior attachment panel 20 and terminating at threaded bore 32 (not shown) of inferior base panel 30.

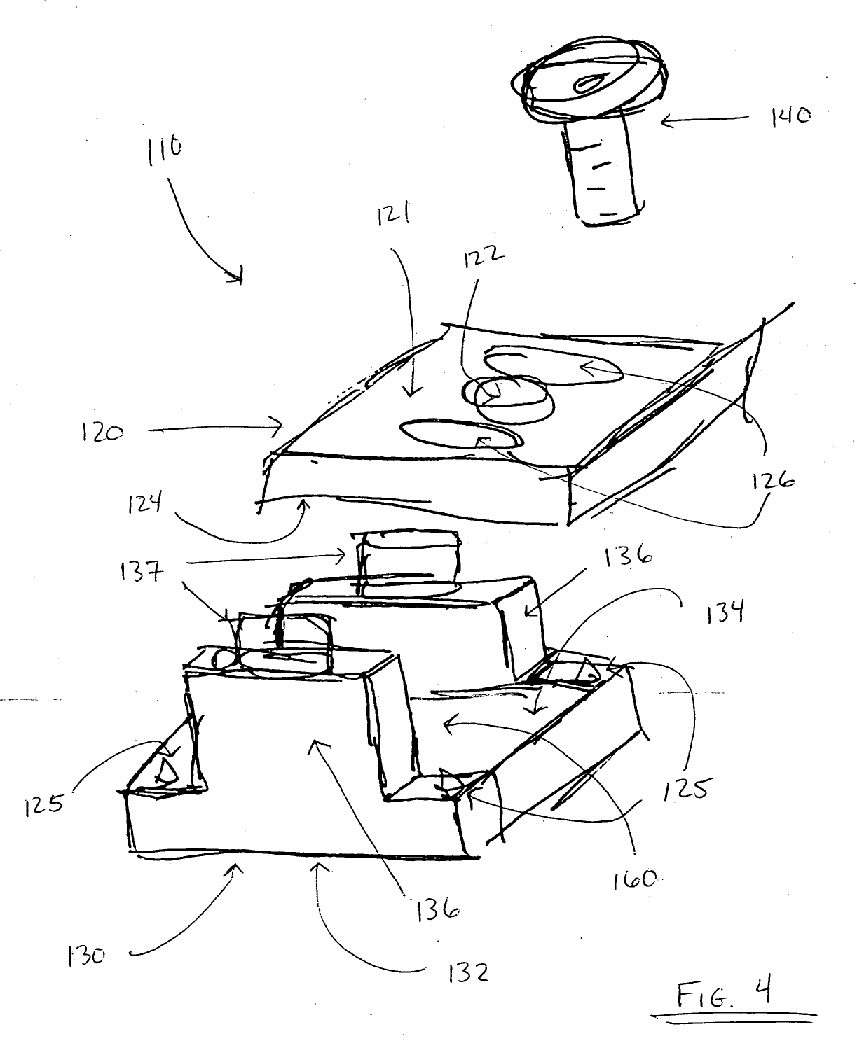

FIG. 4 represents an alternative embodiment 110 of the laminoplasty device 10 of the present invention. The laminoplasty device 110 includes a superior attachment panel 120, an inferior base panel 130, and a coupling screw 140. The superior attachment peel 120 includes a bore 122 that, in a preferred embodiment, includes threads (not shown). Bore 122 is located in a relatively central location of superior latitudinal surface 121 of superior attachment panel 120. Through bore 122 passes coupling screw 140, which when attached with inferior base panel 130 will securely hold together the fully assembled laminoplasty device 110. Inferior latitudinal surface 124 of superior attachment panel 120 may contain spikes (not shown) located, in a preferred embodiment, at each of four corners of the superior attachment panel 120. It is contemplated that in alternative embodiments of laminoplasty device 110, in which the shape of superior attachment panel 120 is other than rectangular or square, the spikes may be located in positions advantageously dictated by the shape of the panel.

Superior attachment panel 120 may also contain any number of coupling orifices 126, to mate with coupling projections 137 on inferior base panel 130. In a preferred embodiment, the number of coupling orifices is two, and they are located to either side of the bore 122, however it is contemplated that the number and location of coupling orifices may vary by design. When assembled with inferior base panel 130, coupling orifices 126 function to prevent lateral movement of superior attachment panel 120. Coupling orifices 126 may be of varying diameter, but must be consistent with the diameter of coupling projections 137.

Inferior base panel 130 includes a threaded bore 132 (not shown). Threaded bore 132 is located along the same longitudinal plane as bore 122 of superior attachment panel 120, in order to effectively accommodate coupling screw 140. Through bore 132 passes coupling screw 140, which when attached with superior attachment panel 120 will securely hold together the fully assembled laminoplasty device 110. Superior latitudinal surface 134 of inferior base panel 130 contains spikes 125 located, in a preferred embodiment, at each of four corners of inferior base panel 130. It is contemplated that in alternative embodiments of laminoplasty device 110, in which the shape of inferior base panel 130 is other than rectangular or square, spikes 125 may be located in positions advantageously dictated by the shape of the panel.

Attached to and extending perpendicularly from superior latitudinal surface 134 of inferior base panel 130 are a pair of fusion corridor walls 136. Fusion corridor walls 136 are comprised of two opposing solid planar walls 136. Inferior base panel 130 forms the floor of fusion corridor 160, while the ceiling is open until coupled with superior attachment panel 120, at which point said superior attachment panel 120 will form the ceiling of fusion corridor 160. Fusion corridor walls 136 may contain any number of coupling projections 137 extending perpendicularly from the top of fusion corridor walls 136. Though the number, size, and location of coupling projections 137 may vary, they must be consistent with the number, size, and location of coupling orifices 126 on superior attachment panel 120. When the laminoplasty device 110 of the present invention is fully assembled, coupling projections 137 will fit securely inside coupling orifices 126, attaching superior attachment panel 120 and inferior base panel 130 in the proper alignment, and forming the completed fusion corridor 160.

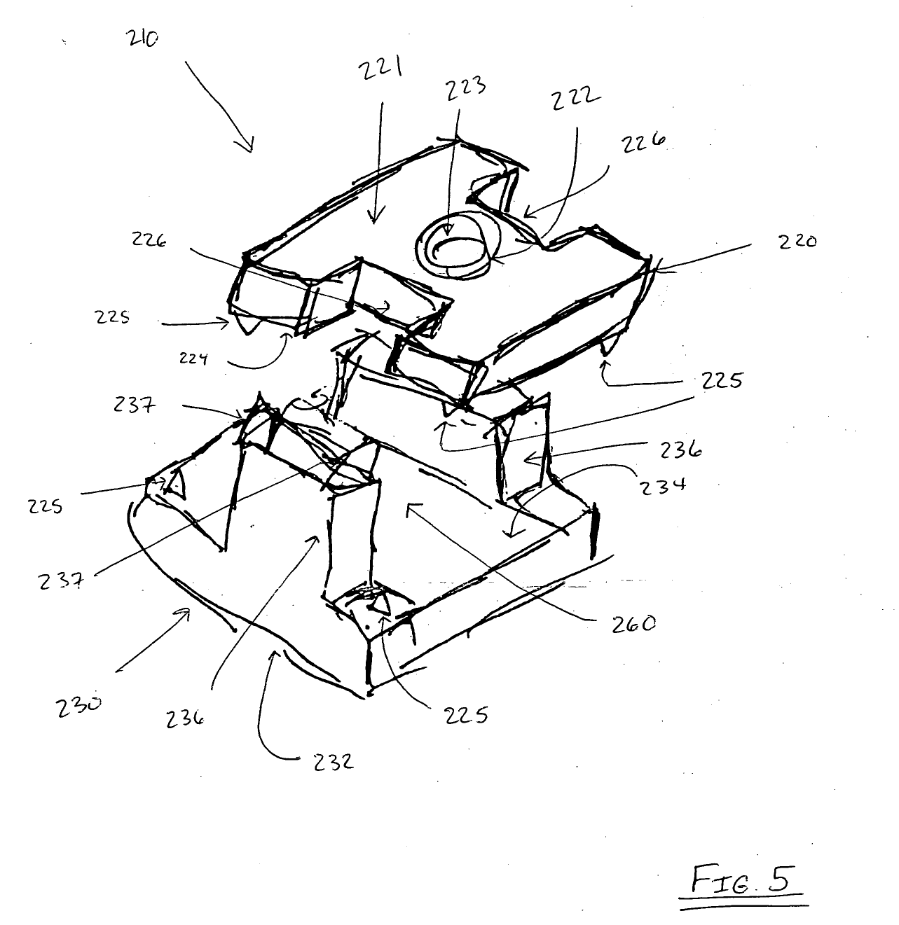

FIG. 5 represents an alternative embodiment 210 of the laminoplasty device 10 of the present invention. The laminoplasty device 210 includes a superior attachment panel 220, an inferior base panel 230, and a coupling screw 240 (not shown). The superior attachment panel 220 includes a bore 222 that, in a preferred embodiment, includes threads 223. Bore 222 is located in a relatively central location of superior latitudinal surface 221 of superior attachment panel 220. Through bore 222 passes coupling screw 240 (not shown), which when attached with inferior base panel 230 will securely hold together the fully assembled laminoplasty device 210. Inferior latitudinal surface 224 of superior attachment panel 220 may contain spikes 225 located, in a preferred embodiment, at each of four corners of the superior attachment panel 220. It is contemplated that in alternative embodiments of laminoplasty device 210, in which the shape of superior attachment panel 220 is other than rectangular or square, the spikes may be located in positions advantageously dictated by the shape of the panel.

Superior attachment panel 220 may also contain any number of coupling indentations 226, to mate with coupling projections 236 on inferior base panel 230. In a preferred embodiment, the number of coupling indentations is two, and they are located to either side of the bore 222, however it is contemplated that the number and location of coupling indentations may vary by design. When assembled with inferior base panel 230, coupling indentations 226 function to prevent lateral movement of superior attachment panel 220. Coupling indentations 226 may be of varying width, but must be consistent with the width of coupling projections 237.

Inferior base panel 230 includes a threaded bore 232 (not shown). Threaded bore 232 is located along the same longitudinal plane as bore 222 of superior attachment panel 220, in order to effectively accommodate coupling screw 240 (not shown). Through bore 232 passes coupling screw 240 (not shown), which when attached with superior attachment panel 220 will securely hold together the fully assembled laminoplasty device 210. Superior latitudinal surface 234 of inferior base panel 230 contains spikes 225 located, in a preferred embodiment, at each of four corners of inferior base panel 230. It is contemplated that in alternative embodiments of laminoplasty device 210, in which the shape of inferior base panel 230 is other than rectangular or square, spikes 225 may be located in positions advantageously dictated by the shape of the panel.

A pair of fusion corridor walls 236 are attached to and extend perpendicularly from superior latitudinal surface 234 of inferior base panel 230. Fusion corridor walls 236 are comprised of two opposing solid planar walls 236. Inferior base panel 230 forms the floor of fusion corridor 260, while the ceiling is open until coupled with superior attachment panel 220, at which point said superior attachment panel 220 forms the ceiling of fusion corridor 260. Fusion corridor walls 236 may contain any number of coupling projections 237 extending perpendicularly from the top of fusion corridor walls 236. Though the number, size, and location of coupling projections 237 may vary, they must be consistent with the number, size, and location of coupling indentations 226 on superior attachment panel 220. When the laminoplasty device 210 of the present invention is fully assembled, coupling projections 236 will fit securely inside coupling indentations 226, attaching superior attachment panel 220 and inferior base panel 230 in the proper alignment, and forming the completed fusion corridor 260.

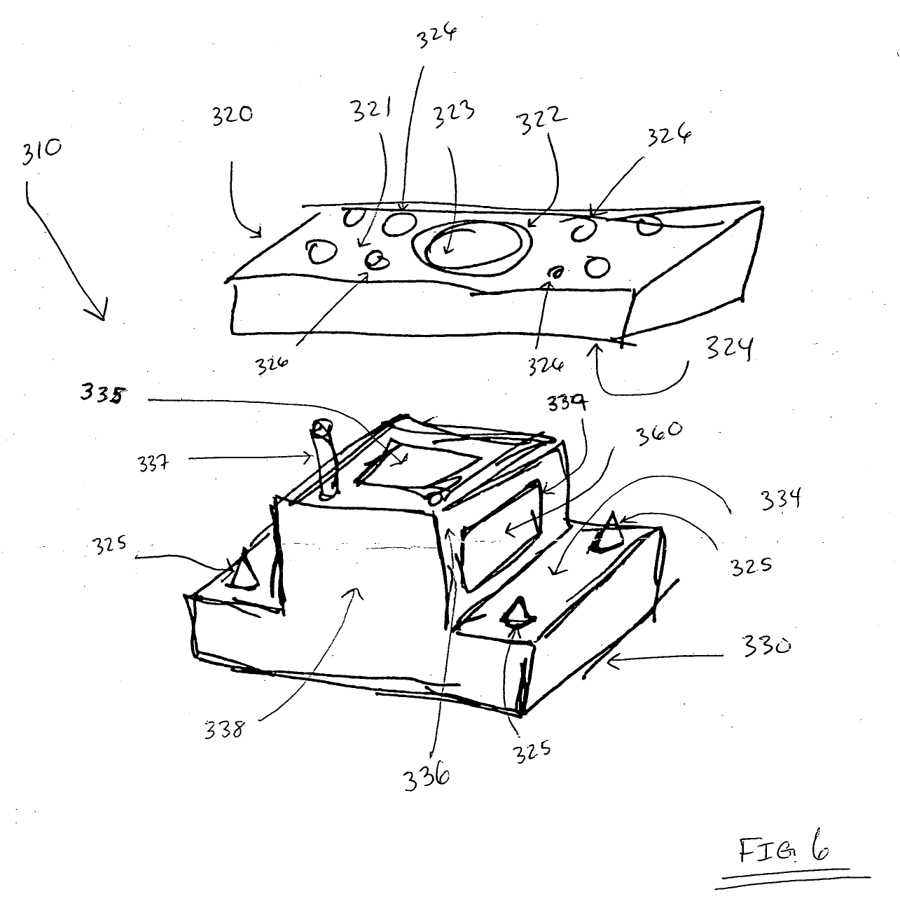

FIG. 6 illustrates yet another embodiment 310 of the laminoplasty device 10 of the present invention. The laminoplasty device 310 includes a superior attachment panel 320, an inferior base panel 330, and a coupling screw 340 (not shown). The superior attachment panel 320 is includes a bore 322 that, in a preferred embodiment, includes threads 323. Bore 322 is located in a relatively central location of superior latitudinal surface 321 of superior attachment panel 320. Through bore 322 passes coupling screw 340 (not shown), which when attached with inferior base panel 330 will securely hold together the fully assembled laminoplasty device 310. Inferior latitudinal surface 324 of superior attachment panel 320 contains spikes 325 (not shown) located, in a preferred embodiment, at each of four corners of superior attachment panel 320. It is contemplated that in alternative embodiments of laminoplasty device 310, in which the shape of superior attachment panel 320 is other than rectangular or square, spikes 325 may be located in positions advantageously dictated by the shape of the panel.

Superior attachment panel 320 may also contain any number of coupling orifices 326, to mate with coupling projections 336 on inferior base panel 330. In a preferred embodiment, the number of coupling orifices is four, and they are located to either side of bore 322, however it is contemplated that the number and location of coupling orifices may vary by design. When assembled with inferior base panel 330, coupling orifices 326 function to prevent lateral movement of superior attachment panel 320. Coupling orifices 326 may be of varying diameter, but must be consistent with the diameter of coupling projections 337.

Inferior base panel 330 includes a threaded bore 332 (not shown). Threaded bore 332 is located along the same longitudinal plane as bore 322 of superior attachment panel 320, in order to effectively accommodate coupling screw 340 (not shown). Through bore 332 passes coupling screw 340 (not shown), which when attached with superior attachment panel 320 will securely hold together the fully assembled laminoplasty device 310. Superior latitudinal surface 334 of inferior base panel 330 contains spikes 325 located, in a preferred embodiment, at each of four corners of the inferior base panel 330. It is contemplated that in alternative embodiments of laminoplasty device 310, in which the shape of the inferior base panel 330 is other than rectangular or square, spikes 325 may be located in positions advantageously dictated by the shape of the panel.

Attached to and extending perpendicularly from superior latitudinal surface 334 of inferior base panel 330 is fusion corridor housing 336. Fusion corridor housing 336 is a four-sided structure comprised of two opposing lateral corridor windows 339 and two opposing solid planar walls 338. Inferior base panel 330 forms the floor of fusion corridor housing 336, while the ceiling contains a vertical corridor window 335 to allow coupling screw 340 (not shown) to pass through. Shown in a square or cubic orientation, it is contemplated that fusion corridor housing 336 could maintain a rectangular or other shape determined in part by the shape of inferior base panel 330. Fusion corridor housing 336 may contain any number of coupling projections 337 extending perpendicularly from the top of fusion corridor housing 336. Though the number, size, and location of coupling projections 337 may vary, they must be consistent with the number, size, and location of coupling orifices 326 on superior attachment panel 320. When the laminoplasty device 310 of the present invention is fully assembled, coupling projections 337 will fit securely inside coupling orifices 326, attaching superior attachment panel 320 and inferior base panel 330 in the proper alignment.

Referring back to FIG. 1, the previously described preferred embodiment of laminoplasty device 10 indicates that bore 22 in superior attachment panel 20 contains threads 23, and inferior base panel 30 contains threaded bore 32. Thus, in the preferred embodiment, both bore 22 and threaded bore 32 contain threads for securing coupling screw 40. It is contemplated that it may be advantageous for one of the aforementioned bores to be smooth rather than threaded. Thus, in alternative embodiments in which coupling screw 40 is inserted in a superior to inferior direction (that is, passing first through bore 22 and then through threaded bore 32), bore 22 may or may not contain threads. Similarly, in alternative embodiments in which coupling screw 40 is inserted in an inferior to superior direction, bore 32 may be without threads, but then bore 22 would be threaded in order to secure coupling screw 40. This alternative approach to securing coupling screw 40 is intended to also apply to the corresponding bores 122, 132, 222, 232, 322, 332 and screws 140, 240, 340 in respective alternative embodiments 110, 210, and 310, and as illustrated in FIGS. 4-6.

1. A device for restoring the structural integrity of bone, comprising:
a first shaped attachment panel having a first surface, a second surface, and a measurable thickness therebetween;
a second shaped attachment panel having a first surface, a second surface, and a measurable thickness therebetween;
at least one of said first and second shaped attachment panels including a housing structure to receive a volume of material to facilitate regeneration of bone; and
at least one locking element dimensioned to secure said first attachment panel to said second attachment panel such that said first and second attachment panels are prevented from moving relative to one another.

2. The device of claim 1, wherein said first shaped attachment panel includes at least one aperture extending therethrough.

3. The device of claim 2, wherein said at least one aperture is threaded.

4. The device of claim 1, wherein said second shaped attachment panel includes at least one aperture extending therethrough.

5. The device of claim 4, wherein said at least one aperture is threaded.

6. The device of claim 1, wherein said second surface of said first shaped attachment panel further includes a plurality of antimigration features.

7. The device of claim 6, wherein said plurality of antimigration features comprises a plurality of spikes.

8. The device of claim 1, wherein said housing structure includes at least a first pair of opposing generally planar surfaces extending generally perpendicularly from said second surface.

9. The device of claim 8, wherein said housing structure further includes a second pair of opposing generally planar surfaces extending generally perpendicularly from said second surface.

10. The device of claim 8, wherein said first pair of opposing generally planar surfaces comprises a pair of opposing solid surfaces.

11. The device of claim 9, wherein said second pair of opposing generally planar surfaces each include an aperture extending therethrough.

12. The device of claim 1, wherein said second surface of said second shaped attachment panel further includes a plurality of antimigration features.

13. The device of claim 12, wherein said plurality of antimigration features comprises a plurality of spikes.

14. The device of claim 1, wherein said locking mechanism comprises a threaded screw.

15. A system for restoring the structural integrity of bone, comprising:
a device for facilitating bone fusion, said device including a first shaped attachment panel having a first surface, a second surface, and a measurable thickness therebetween, a second shaped attachment panel having a first surface, a second surface, and a measurable thickness therebetween, said second shaped attachment panel including a housing structure having at least a first pair of opposing generally planar surfaces extending generally perpendicularly from said second surface, and at least one leaking element dimensioned to secure said first attachment panel to said second attachment panel such that said first and second attachment panels are prevented from moving relative to one another; and
a volume of bone fusion material for insertion into said device.

16. The system of claim 15, wherein said first shaped attachment panel includes at least one aperture extending therethrough.

17. The system of claim 16, wherein at least one aperture is threaded.

18. The system of claim 15, wherein said second shaped attachment panel includes at least one aperture extending therethrough.

19. The system of claim 18, wherein at least one aperture is threaded.

20. The system of claim 15, wherein said second surface of said first shaped attachment panel further includes a plurality of antimigration features.

21. The system of claim 20, wherein said plurality of antimigration features comprises a plurality of spikes.

22. The system of claim 15, wherein said second surface of said first shaped attachment panel further includes a housing structure, said housing structure having a first pair of opposing generally planar surfaces and a second pair of opposing generally planar surfaces extending generally perpendicularly from said second surface.

23. The system of claim 22, wherein said first pair of opposing generally planar surfaces comprises a pair of opposing solid surfaces.

24. The system of claim 22, wherein said second pair of opposing generally planar surfaces each include an aperture extending therethrough.

25. The system of claim 15, wherein said second surface of said second shaped attachment panel further includes a plurality of antimigration features.

26. The system of claim 25, wherein said plurality of antimigration features comprises a plurality of spikes.

27. The system of claim 15, wherein said housing structure of said second shaped attachment panel includes a second pair of opposing generally planar surfaces extending generally perpendicularly from said second surface.

28. The system of claim 27, wherein said second pair of opposing planar surfaces each include an aperture extending therethrough.

29. The system of claim 15, wherein said locking mechanism comprises a threaded screw.

30. The system of claim 15, wherein said bond fusion material comprises bone grafted from another part of a patient's body, including but not limited to the hip bone.

31. A method for restoring the structural integrity of bone, comprising:
providing a device for facilitating bone fusion, said device including a first shaped attachment panel having a first surface, a second surface, and a measurable thickness therebetween, a second shaped attachment panel having a first surface, a second surface, and a measurable thickness therebetween, said second shaped attachment panel including a housing structure having at least a first pair of opposing generally planar surfaces extending generally perpendicularly from said second surface, and at least one locking element dimensioned to secure said first attachment panel to said second attachment panel such that said first and second attachment panels are prevented from moving relative to one another;
providing a volume of bone fusion material for insertion into said device; and
implanting said device into an aperture formed in a bone of a patient.
